(19) United States
(12) Patent Application Publication
(10) Pub. No.: US 2025/0107782 A1
(43) Pub. Date: Apr. 3, 2025

(54) ULTRASOUND IMAGING APPARATUS AND CONTROL METHOD THEREFOR, ULTRASOUND IMAGING SYSTEM, AND STORAGE MEDIUM

(71) Applicant: GE Precision Healthcare LLC, Waukesha, WI (US)

(72) Inventor: Haihua Miao, Wuxi (CN)

(21) Appl. No.: 18/893,735

(22) Filed: Sep. 23, 2024

(30) Foreign Application Priority Data
Sep. 28, 2023 (CN) 202311281480.9

(51) Int. Cl. A61B 8/00

(52) U.S. Cl. CPC A61B 8/565

(57) ABSTRACT

The method of controlling a ultrasound system includes establishing a communication connection with an external device; transmitting ultrasound data to the external device via the communication connection; receiving, via the communication connection, user interface information about the ultrasound data, the user interface information being generated on a display interface of the external device; and in response to generation of a switching instruction, switching an input device of the ultrasound imaging apparatus to control a user interface of the external device.

### CROSS-REFERENCE TO RELATED APPLICATION

This application claim priority to Chinese Patent Application No. 202311281480.9, which was file on Sep. 28, 2023 at the Chinese Patent Office. The entire contents of the above-listed application are incorporated by reference herein in their entirety.

### TECHNICAL FIELD

Embodiments of the present application relate to the technical field of medical devices, and in particular to an ultrasound imaging apparatus and a control method therefor, an ultrasound imaging system, and a storage medium.

### BACKGROUND

With the development of ultrasound imaging technology, ultrasound imaging has become widely used in the medical field. A physician examines a subject under examination using an ultrasound imaging device to acquire ultrasound data, and performs medical diagnosis on the subject under examination on the basis of the ultrasound data.

In recent years, with the development of network technology, telemedicine has also been rapidly promoted. For example, a physician can remotely interact with other personnel via an external device while performing ultrasound scanning, thereby achieving efficient sharing of medical resources.

It should be noted that the above introduction of the background is only for the convenience of clearly and completely describing the technical solutions of the present application, and for the convenience of understanding for those skilled in the art.

### SUMMARY

The inventors have found that a physician performing telemedicine needs to operate an external device to interact with remote personnel, but an operating component of the external device is not generally provided on an ultrasound imaging device. In addition, the physician generally further needs to operate the ultrasound imaging device, and the operation comprises operating a scanning probe, various buttons and keys on the ultrasound imaging device, and the like, so that it is difficult for the physician operating the ultrasound imaging device to directly operate the external device due to distance, resulting in inconvenience.

To address at least one of the above technical problems, embodiments of the present application provide an ultrasound imaging apparatus and a control method therefor, an ultrasound imaging system, and a storage medium.

According to a first aspect of the embodiments of the present application, a control method for an ultrasound imaging apparatus is provided. The method comprises:
Establishing a communication connection with an external device;
Transmitting ultrasound data to the external device via the communication connection;
Receiving, via the communication connection, user interface information about the ultrasound data, the user interface information being generated on a display interface of the external device; and
In response to generation of a switching instruction, switching an input device of the ultrasound imaging apparatus to control a user interface of the external device.

In one or more embodiments, the generation of the switching instruction is based on at least one of the following means:
Moving a cursor of the input device on a display apparatus of the ultrasound imaging apparatus out of an edge of the display apparatus towards the external device;
Operating a switch button in the input device; and
Determining, using an image capture apparatus, that a line of sight of a user is directed to the external device.

In one or more embodiments, determining, using an image capture apparatus, that a line of sight of a user is directed to the external device comprises:
Acquiring the line of sight of the user using the image capture apparatus, and
When it is determined that the line of sight is directed to the external device, causing the input device to control the external device.

In one or more embodiments, determining, using an image capture apparatus, that a line of sight of a user is directed to the external device further comprises:
When it is determined that the line of sight is not directed to the external device, causing the input device to control the ultrasound imaging apparatus.

In one or more embodiments, the input device comprises at least one of a trackball, a keyboard, a touch pad, and a touch screen.

In one or more embodiments, the input device comprises at least one of a trackball and a touch pad, and the input device controlling the user interface of the external device comprises:
Determining a controllable region of the user interface of the external device, and restricting a cursor corresponding to the trackball or the touch pad within the controllable region.

In one or more embodiments, the input device comprises a touch screen, and the touch screen controlling the user interface of the external device comprises:
Projecting interface information of the external device onto the touch screen.

In one or more embodiments, the display interface of the external device is configured to be touch-operable.

In one or more embodiments, the external device comprises an image capture apparatus.

According to a second aspect of the embodiments of the present application, an ultrasound imaging apparatus is provided. The ultrasound imaging apparatus comprises an input device and a processor, and the processor is configured to execute the method according to the embodiments of the first aspect.

According to a third aspect of the embodiments of the present application, an ultrasound imaging system is provided. The ultrasound imaging system comprises the ultrasound imaging apparatus according to the embodiments of the second aspect and an external device connected to the ultrasound imaging apparatus.

According to a fourth aspect of the embodiments of the present application, a non-transitory computer-readable storage medium for storing a computer program is provided, and when executed by a computer, the computer program causes the computer to execute the method according to the embodiments of the first aspect.

One of the beneficial effects of the embodiments of the present application is that: switching a control object of the input device of the ultrasound imaging apparatus by means of a switching instruction enables the input device of the ultrasound imaging apparatus to control an external device, thereby improving convenience and work efficiency.

With reference to the following description and drawings, specific implementations of the embodiments of the present application are disclosed in detail, and the means by which the principles of the embodiments of the present application can be employed are illustrated. It should be understood that the embodiments of the present application are therefore not limited in scope. Within the scope of the spirit and clauses of the appended claims, the embodiments of the present application include many changes, modifications, and equivalents.

### BRIEF DESCRIPTION OF THE DRAWINGS

The included drawings are used to provide further understanding of the embodiments of the present application, which constitute a part of the description and are used to illustrate the implementations of the present application and explain the principles of the present application together with textual description. Evidently, the drawings in the following description are merely some embodiments of the present application, and a person of ordinary skill in the art may obtain other implementations according to the drawings without involving inventive effort. In the drawings:

FIG. 1 is a schematic diagram of an ultrasound imaging system according to an embodiment of the present application;

FIG. 2 is a schematic diagram of a control method for an ultrasound imaging apparatus according to an embodiment of the present application;

FIG. 3 is another schematic diagram of a control method for an ultrasound imaging apparatus according to an embodiment of the present application;

FIG. 4 is another schematic diagram of an ultrasound imaging system according to an embodiment of the present application; and

FIG. 5 is a schematic diagram of an ultrasound imaging apparatus according to an embodiment of the present application.

### DETAILED DESCRIPTION

The foregoing and other features of the embodiments of the present application will become apparent from the following description with reference to the drawings. In the description and drawings, specific implementations of the present application are disclosed in detail, and part of the implementations in which the principles of the embodiments of the present application may be employed are indicated. It should be understood that the present application is not limited to the described implementations. On the contrary, the embodiments of the present application include all modifications, variations, and equivalents which fall within the scope of the appended claims.

In the embodiments of the present application, the terms “first”, “second”, etc., are used to distinguish different elements, but do not represent a spatial arrangement or temporal order, etc., of these elements, and these elements should not be limited by these terms. The term “and/or” includes any and all combinations of one or more associated listed terms. The terms “comprise”, “include”, “have”, etc., refer to the presence of described features, elements, components, or assemblies, but do not exclude the presence or addition of one or more other features, elements, components, or assemblies.

In the embodiments of the present application, the singular forms “a” and “the”, etc., include plural forms, and should be broadly construed as “a type of” or “a class of” rather than being limited to the meaning of “one”. Furthermore, the term “the” should be construed as including both the singular and plural forms, unless otherwise specified in the context. In addition, the term “according to” should be construed as “at least in part according to . . . ” and the term “on the basis of” should be construed as “at least in part on the basis of . . . ”, unless otherwise specified in the context.

The features described and/or illustrated for one implementation may be used in one or more other implementations in the same or similar manner, be combined with features in other embodiments, or replace features in other implementations. The terms “include/comprise” when used herein refer to the presence of features, integrated components, steps, or assemblies, but do not preclude the presence or addition of one or more other features, integrated components, steps, or assemblies.

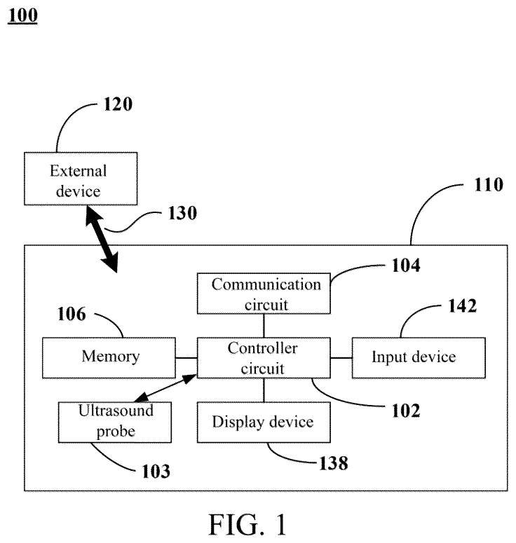

Embodiments of the present application provide an ultrasound imaging system. FIG. 1 is a schematic diagram of an ultrasound imaging system according to an embodiment of the present application.

As shown in FIG. 1, an ultrasound imaging system 100 includes an ultrasound imaging apparatus 110 and an external device 120. The ultrasound imaging apparatus 110 and the external device 120 exchange information via a communication connection 130. The ultrasound imaging apparatus 110 may include a controller circuit 102 and an input device 142. In addition, the ultrasound imaging apparatus 110 may further include an ultrasound probe 103, a communication circuit 104, a display apparatus 138, the input device 142, and a memory 106.

The ultrasound probe 103 may include, for example, elements such as a transducer, a transmitter, a transmit beam former and a detector/SAP electronics, (not shown). The detector/SAP electronics may be used to control the switching of the transducer elements. The detector/SAP electronics may also be used to group the transducer elements into one or more sub-holes. The ultrasound probe 103 may perform wired or wireless communication with the controller circuit 102 to send acquired data to the controller circuit 102, and the controller circuit 102 may process the data acquired by the ultrasound probe 103 to acquire ultrasound data such as an ultrasound image. For implementations of the ultrasound probe 103, reference may be made to the related art, and the embodiments of the present application are not limited thereto.

The controller circuit 102 is configured to control operation of the ultrasound imaging apparatus 110. The controller circuit 102 may include one or more processors. Optionally, the controller circuit 102 may include a central processing unit (CPU), one or more microprocessors, a graphics processing unit (GPU), or any other electronic component capable of processing inputted data according to a specific logic instruction. Optionally, the controller circuit 102 may include and/or represent one or more hardware circuits or circuit systems, and the hardware circuit or circuit system includes, is connected to, or includes and is connected to one or more processors, controllers, and/or other hardware logic-based apparatuses. Additionally or alternatively, the controller circuit 102 may execute an instruction stored on a tangible and non-transitory computer-readable medium (e.g., the memory 106).

The controller circuit 102 may be connected to and/or control the communication circuit 104. The communication circuit 104 is configured to receive and/or transmit information along a bidirectional communication link with one or more alternate external devices 120, etc. The external device 120 may include patient information, a machine learning algorithm, a remotely stored medical image from a previous scan and/or a diagnosis and treatment period of a patient, etc. The communication circuit 104 may represent hardware for transmitting and/or receiving data along a bidirectional communication link. The communication circuit 104 may include a transceiver, a receiver, etc., and an associated circuit system (e.g., an antenna) for communicating (e.g., transmitting and/or receiving) with the one or more alternate external devices 120 by wired and/or wireless means. For example, protocol firmware for transmitting and/or receiving data along a bidirectional communication link may be stored in the memory 106 accessed by the controller circuit 102. The protocol firmware provides network protocol syntax to the controller circuit 102 so as to assemble a data packet, establish and/or segment data received along the bidirectional communication link, and so on.

The bidirectional communication link may be a wired (e.g., by means of a physical conductor) and/or wireless communication (e.g., utilizing a radio frequency (RF)) link for exchanging data (e.g., a data packet) between the one or more alternate external devices 120. The bidirectional communication link may be based on a standard communication protocol, such as Ethernet, TCP/IP, Wi-Fi, 802.11, a customized communication protocol, Bluetooth, etc.

The controller circuit 102 is operatively connected to the display apparatus 138 and the input device 142. The display apparatus 138 may include one or more liquid crystal display apparatuses (e.g., light emitting diode (LED) backlights), organic light emitting diode (OLED) display apparatuses, plasma display apparatuses, CRT display apparatuses, and the like. The display apparatus 138 may display patient information, one or more medical images and/or videos, a graphical user interface, or a component received by the display apparatus 138 from the controller circuit 102, one or more 2D, 3D or 4D ultrasound image data sets from ultrasound data stored in the memory 106, or anatomical measurement, diagnosis, processing information, and the like currently acquired in real time.

The controller circuit 102 may be configured to process the acquired ultrasound data (e.g., RF signal data or IQ data pairs), and prepare and/or generate frames of ultrasound image data representing an anatomical structure of interest so as to display the same on the display apparatus 138. The acquired ultrasound data may be processed by the controller circuit 102 in real time during a scanning or treatment process of ultrasound examination when echo signals are received. Additionally or alternatively, the ultrasound data may be temporarily stored in the memory 106 during a scanning process, and processed in a less real-time manner in a live or off-line operation.

The memory 106 may be used to store processed frames of acquired ultrasound data that are not scheduled to be immediately displayed, or may be used to store post-processed images (e.g., shear wave images and strain images), firmware or software corresponding to, for example, a graphical user interface, one or more default image display settings, programmed instructions, and the like. The memory 106 may store a medical image, such as a 3D ultrasound image data set of ultrasound data, wherein such a 3D ultrasound image data set is accessed to present 2D and 3D images. For example, the 3D ultrasound image data set may be mapped to the corresponding memory 106 and one or more reference planes. Processing of ultrasound data that includes the ultrasound image data set may be based in part on user input, e.g., a user selection received at the input device 142.

The memory 106 includes a parameter, an algorithm, one or more protocols of ultrasound examination, data values, and the like used by the controller circuit 102 to execute one or more operations described in the present application. The memory 106 may be a tangible and non-transitory computer-readable medium such as a flash memory, a RAM, a ROM, an EEPROM, etc. The memory 106 may include a set of learning algorithms (e.g., a convolutional neural network algorithm, a deep learning algorithm, a decision tree learning algorithm, etc.) configured to define an image analysis algorithm. During execution of the image analysis algorithm, the controller circuit 102 is configured to identify a section (or a view or an anatomical plane) of an anatomical structure of interest in a medical image. Optionally, an image analysis algorithm may be received by means of the communication circuit 104 along one among bidirectional communication links, and stored in the memory 106. It can be understood that the anatomical structure of interest may be a specific anatomical feature in a site to be scanned, and may be, for example, a muscle, a blood vessel, a tissue to be subjected to intervention (e.g., a tumor), etc.

The input device 142 controls operation of the controller circuit 102 and the external device 120. The input device 142 is configured to receive an input from a clinician and/or an operator of the ultrasound imaging system 100. The input device 142 may include a keyboard, a mouse, a trackball, a touchpad, one or more physical buttons, and the like. Optionally, the display apparatus 138 may be a touch screen display apparatus that includes at least one part of the input device 142. For example, one part of the input device 142 may correspond to a graphical user interface (GUI) that is generated by the controller circuit 102 and is shown on the display apparatus 138. The touch screen display apparatus may detect the presence of a touch from the operator on the display apparatus 138, and may also identify the location of the touch relative to the surface area of the display apparatus 138. For example, a user may select, by touching or contacting the display apparatus 138, one or more user interface components of the user interface (GUI) shown on the display apparatus. User interface components may correspond to icons, text boxes, menu bars, etc., shown on the display apparatus 138. A clinician may select, control, and use a user interface assembly, interact with the same, and so on, so as to send an instruction to the controller circuit 102 to perform one or more operations described in the present application. For example, a touch may be applied using at least one among a hand, a glove, a stylus, and the like.

The external device 120 may be used to receive ultrasound data, perform remote ultrasound, and the like. The external device 120 includes a communication module for communicating with the ultrasound imaging apparatus 110, and the communication module is, for example, a 4G/5G module, a ZigBee wireless communication module, a wireless Bluetooth module, a NearLink communication module, a WiFi communication module, a customer premises equipment (CPE) communication module, and the like. The external device 120 may exchange ultrasound data and information related to ultrasound data with the ultrasound imaging apparatus 110 via the communication module. The information related to ultrasound data may be, for example, user interface information about the ultrasound data, and that is generated on a display interface of a respective display component on the basis of the ultrasound data, such as coordinates and dimensions of the user interface. The external device 120 may further include one or more image capture apparatuses. The image capture apparatus may be built in the external device 120. For example, the image capture apparatus may be a front-facing camera and/or a rear-facing camera of the external device 120. The image capture apparatus may be external to the external device 120. The image capture apparatus may perform a remote ultrasound function of the external device, and may also perform line-of-sight determination. The remote ultrasound and the line-of-sight determination may be implemented by means of one image capture apparatus, or may be implemented by means of different image capture apparatuses. The remote ultrasound and the line-of-sight determination are described in detail below.

A scanning operator of the ultrasound imaging system 100 may exchange information with remote personnel by means of the external device 120.

While the functions of the various components of the ultrasound imaging system 100 and the manner in which they cooperate with one another have been exemplarily described above, it will be appreciated that those skilled in the art, in light of the above teachings of the present disclosure, may also adjust the arrangement of the ultrasound imaging system 100.

The inventors have found that, during an ultrasound scan, a scanning operator needs to hold an ultrasound probe with one hand and operate various keys, buttons, etc., on an ultrasound imaging apparatus with the other hand, so that when remote communication is performed via an external device, it is difficult for the scanning operator operating the ultrasound imaging device to directly operate the external apparatus due to a distance, and usually, the scanning operator has to put down the ultrasound probe to operate the external device, and needs to pick up the ultrasound probe to perform an ultrasound scanning operation after a current operation of the external device is completed. The scanning operator cannot efficiently operate the external device and perform the ultrasound scanning operation.

The embodiments of the present application provide a control method for an ultrasound imaging apparatus. In the control method, an input device of an ultrasound imaging apparatus is controlled to be capable of performing an input operation on an external device.

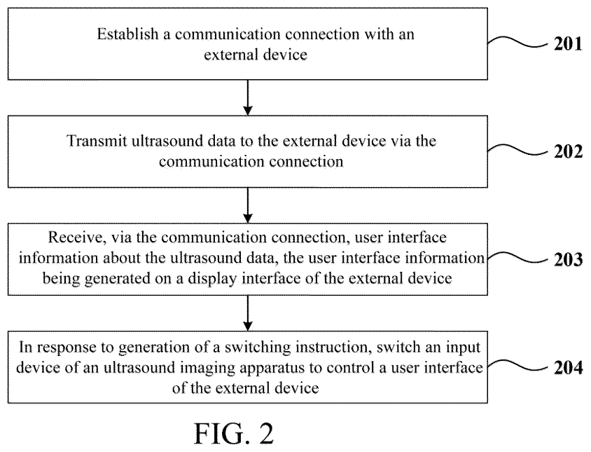

FIG. 2 is a schematic diagram of a control method for an ultrasound imaging apparatus according to an embodiment of the present application.

As shown in FIG. 2, the method includes:
201: Establishing a communication connection with an external device;
202: Transmitting ultrasound data to the external device via the communication connection;
203: Receiving, via the communication connection, user interface information about the ultrasound data, the user interface information being generated on a display interface of the external device; and
204: In response to generation of a switching instruction, switching an input device of the ultrasound imaging apparatus to control a user interface of the external device.

Thus, switching a control object of the input device of the ultrasound imaging apparatus by means of a switching instruction enables the input device of the ultrasound imaging apparatus to control an external device. That is, an ultrasound scanning operation and operation of the external device can be performed using the input device of the ultrasound imaging apparatus, thereby improving convenience and work efficiency.

In the embodiments of the present application, user interface information that is about the ultrasound data and that is generated on the display interface of the external device may be, for example, coordinates, dimensions, etc., of the user interface about the ultrasound data on the display interface of the external device, thereby enabling accurate control of the user interface of the external device.

An application scenario of the embodiments of the present application may be remote ultrasound. That is, a physician at a remote terminal may remotely communicate with a scanning operator of an ultrasound imaging apparatus to instruct the scanning operator to perform ultrasound scanning, or the scanning operator and the physician at the remote terminal share ultrasound data. For example, in the ultrasound imaging system 100 shown in FIG. 1, a scanning operator may operate the input device of the ultrasound imaging apparatus 110 to acquire an ultrasound image. The ultrasound imaging apparatus 110 transmits the ultrasound data such as the ultrasound image to the external device 120. The external device 120 may share the ultrasound data with a physician at a remote terminal via, for example, a 5G module. The physician and the scanning operator interact with each other via the external device 120, and the scanning operator may operate the external device 120 to obtain guidance of the remote physician and follow the guidance of the physician to perform ultrasound scanning on a patient in real time. Thus, for example, the remote physician can determine required ultrasound data in real time according to the scanning condition of the patient, and the scanning operator under the guidance of the physician can quickly and accurately acquire data required for disease diagnosis, thereby improving diagnosis efficiency. In another aspect, even if the scanning operator of the ultrasound imaging apparatus has no medical knowledge of disease diagnosis, ultrasound data required for disease diagnosis can still be efficiently acquired, thereby avoiding waste of medical resources caused by acquisition of useless data by the scanning operator.

Another application scenario of the embodiments of the present application may be that a scanning operator communicates with a remote trainee via an external device to remotely teach the trainee knowledge related to ultrasound scanning. For example, in the ultrasound imaging system 100 shown in FIG. 1, the input device of the ultrasound imaging apparatus 110 is operated to acquire an ultrasound image. The ultrasound imaging apparatus 110 transmits ultrasound data such as the ultrasound image to the external device 120. The external device 120 may send the ultrasound data to or share the ultrasound data with a trainee at a remote terminal. In addition, when the external device 120 has an image capture apparatus, an operation video of a scanning operator may be captured by using the image capture apparatus of the external device 120, and may be transmitted to the trainee at the remote terminal in real time. The scanning operator may teach the trainee at the remote terminal knowledge related to a previous ultrasound scanning operation by operating the external device 120 while performing an ultrasound scanning operation.

It should be understood by those skilled in the art that the above description of application scenarios is to make the implementation of the embodiments of the present application clearer, and should not be construed as limiting the embodiments of the present application. The embodiments of the present application can also be applied to other scenarios, and application scenarios are not limited by the embodiments of the present application.

In some embodiments, the generation of the switching instruction is based on at least one of the following means:
Moving a cursor of the input device on a display apparatus of the ultrasound imaging apparatus out of an edge of the display apparatus towards the external device;
Operating a switch button in the input device; and
Determining, using an image capture apparatus, that a line of sight of a user is directed to the external device.

Therefore, the user can flexibly and quickly perform switching between the operation of the ultrasound imaging apparatus and the operation of the external device via a simple operation.

For example, in the ultrasound imaging system 100 shown in FIG. 1, the input device 142 may include at least one of a trackball, a keyboard, a touch pad, and a touch screen.

In some embodiments, the input device 142 includes a trackball and/or a touch pad. When the cursor of the input device 142 on the display apparatus 138 moves to an edge of the display apparatus 138 towards the external device 120, this indicates that the operation of the current apparatus (i.e., the ultrasound imaging apparatus 110) by the operator temporarily ends, and that switching to the display interface of the external device 120 needs to be performed to perform an operation; therefore a switching instruction is generated.

In the above example, operation 204 may include: determining a controllable region of the user interface of the external device 120, and restricting a cursor corresponding to the trackball or the touch pad within the controllable region. For example, dimensions of the user interface of the external device 120 are different from dimensions of the display interface of the display apparatus 138 of the ultrasound imaging apparatus, and a region dimensionally similar to the display interface of the display apparatus 138 may be defined on the user interface of the external device 120. The interface related to the ultrasound data may be displayed in the region, and the cursor corresponding to the trackball or the touch pad may be restricted within the region, thereby facilitating switching of data such as an ultrasound image between the two display interfaces.

Alternatively, the user interface of the external device 120 displays some information that cannot be changed or is not allowed to be changed, for example, information related to privacy information of a patient, or information related to the external device 120, so that a region on the user interface other than a region related to said information is determined to be a controllable region, thereby preventing the user from misoperating said information on the external device 120.

In some embodiments, the input device 142 includes a touch screen, and operation 204 may include: projecting interface information of the external device onto the touch screen. Thus, the interface of the external device is controlled by controlling the touch screen.

As another example, in the ultrasound imaging system 100 shown in FIG. 1, the input device 142 may be further provided with a switch button used to trigger the switching instruction. The switch button may be a hardware button or a software button. When switching of the control object is required, the user triggers the button. In response to the trigger operation, a switching instruction is generated to switch to control the user interface of the external device or control the ultrasound imaging apparatus.

As another example, the ultrasound imaging system 100 shown in FIG. 1 may further include an image capture apparatus. The image capture apparatus detects that a line of sight of the operator is directed to the external device 120, and this indicates that the operator is interested in the external device 120, so that a switching instruction is generated. In addition, alternatively, the external device 120 may include the image capture apparatus that detects the direction of the line of sight of the operator, or the image capture apparatus may be provided on the ultrasound imaging apparatus.

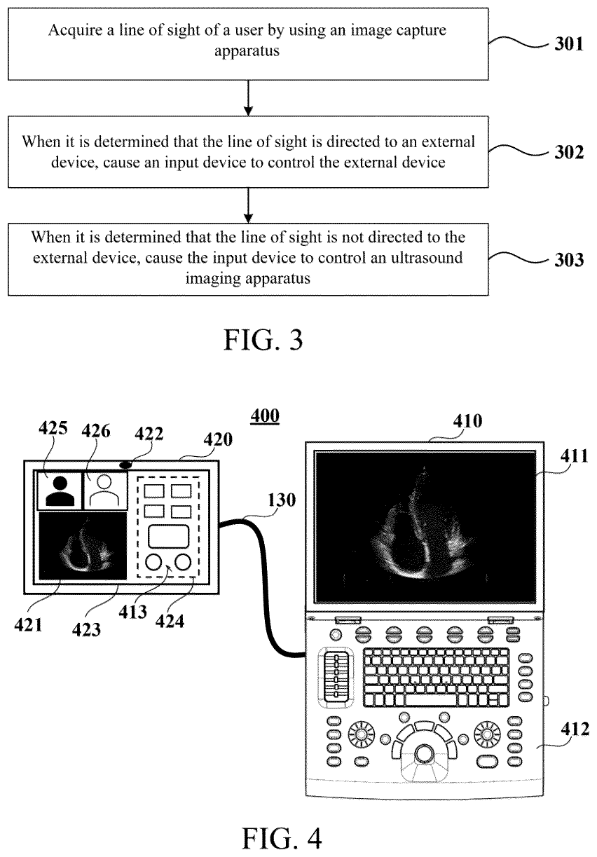

FIG. 3 is another schematic diagram of a control method for an ultrasound imaging apparatus according to an embodiment of the present application. FIG. 3 shows steps of switching the control object of the input device on the basis of the direction of the line of sight.

As shown in FIG. 3, the method includes:
301: Acquiring the line of sight of the user using the image capture apparatus, and
302: When it is determined that the line of sight is directed to the external device, causing the input device to control the external device.

In the embodiments of the present application, for example, the image capture apparatus acquires a face image of a user, and analyzes the acquired face image on the basis of a neural network model to obtain a line of sight of the user. Other methods may also be used to acquire the line of sight of the user. For details, reference may be made to the related art, and the embodiments of the present application do not limit how to acquire the line of sight of the user.

In the embodiments of the present application, for example, the face image of the user may be analyzed on the basis of a deep learning model to acquire a head posture. A point of regard of the user may be calculated according to parameters such as the head posture, the angle of the line of sight, a distance from the user to the external device, etc. When the point of regard of the user falls within a predetermined region where the external device is located, it is determined that the line of sight of the user is directed to the external device. Other methods may also be used to determine the direction of the line of sight. For details, reference may be made to the related art, and the embodiments of the present application do not limit how to determine the direction of the line of sight.

In addition, as shown in FIG. 3, the method may further include:
303: When it is determined that the line of sight is not directed to the external device, causing the input device to control the ultrasound imaging apparatus.

Therefore, the user can switch the control object of the input device by switching only the line of sight, thereby further facilitating switching between operation of the ultrasound imaging apparatus and operation of the external device.

However, the present application is not limited thereto. For example, the image capture apparatus may also acquire a posture and/or an action of another part of the scanning operator to generate a switching instruction. For example, when an action or a posture of the scanning operator raising a hand is acquired via the image capture apparatus, a switching instruction is generated. In addition, a switching instruction may be generated by combining posture information of the operator acquired by the image capture apparatus. For example, when the image capture apparatus sequentially acquires information indicating that the line of sight of the scanning operator is directed to the external device and information indicating that a hand of the scanning operator is raised, a switching instruction is generated, thereby improving operation reliability.

In some embodiments, the display interface of the external device is configured to be touch-operable. For example, in the ultrasound imaging system 100 shown in FIG. 1, the external device 120 may be a portable computer, such as a tablet computer or the like, on which a touch screen operation can be performed.

As can be seen from the above embodiments, switching the control object of the input device of the ultrasound imaging apparatus via the switching instruction enables the input device of the ultrasound imaging apparatus to control the external device, thereby improving convenience and work efficiency.

In order to further describe the embodiments of the present application, referring to FIG. 4, FIG. 4 shows a schematic diagram of an ultrasound imaging system 400 according to some embodiments of the present application.

The ultrasound imaging system 400 includes an ultrasound imaging apparatus 410 and an external device 420. There is a communication connection 130 between the ultrasound imaging apparatus 410 and the external device 420. The communication connection 130 enables data exchange and transmission of control instructions between the ultrasound imaging apparatus 410 and the external device 420. The communication connection 130 can be configured with a variety of functions. In one example, the communication connection 130 can allow data of the ultrasound imaging apparatus 410 to be transmitted to the external device 420. As shown in FIG. 4, an ultrasound image 411 of the ultrasound imaging apparatus 410 is transmitted to the external device 420 via the communication connection 130, and the external device 420 can display the same image 421 on a user interface 423 thereof. In a usage scenario of remote ultrasound, a screen of the external device 420 can be shared to a remote terminal by means of telecommunication technology, and a remote user can learn the state of the current ultrasound scan via the image 421, thereby learning or providing guidance.

Furthermore, in some examples, the external device 420 includes an image capture apparatus 422. The image capture apparatus 422 can acquire required image information. There are a plurality of types of image information. For example, the image information may be line-of-sight and/or face information of the user. In a typical usage scenario, it is difficult for the user operating the ultrasound imaging apparatus 410 to operate the external device 420 due to distance. In the manner described in the above embodiments of the present application, the line-of-sight information of the user acquired by the image capture apparatus 422 can be received by a processor of the ultrasound imaging apparatus 410 via the communication connection 130, so as to determine whether the attention of the user is currently focused on the external device 420. If so, an input instruction capable of controlling an input device 412 is configured to be sent to the external device 420 to control the external device 420. In such configurations, during an ultrasound scan, the user can freely control the ultrasound imaging apparatus 410 and the external device 420 simultaneously by means of the input device 412 of the ultrasound imaging apparatus 410 without adjusting position or performing many additional operations.

The external device 420 may be controlled via a key, a trackball, and a touch pad (not shown in FIG. 4) on the input device 412. In one example, for a controllable region 424 (represented by a dashed box) of the user interface 423 of the external device 420, a cursor 413 corresponding to the trackball (or the touch pad) of the input device 412 may be restricted within the controllable region 424. In this way, the cursor 413 is operated more quickly. Especially when that the user interface is large and is complex in composition, it is not too difficult to control the cursor 413 to reach a desired position. The above embodiments can ensure accuracy and efficiency of control even if the external device 420 is controlled using the input device 412 of the ultrasound imaging apparatus 410. The controllable region refers to a functional region on the user interface that can be operated by means of a user input. FIG. 4 shows one complete controllable region 424. However, it can be understood that under the teachings of the embodiments of the present application, a plurality of discrete controllable regions 424 may be present on the user interface. Furthermore, the controllable region 424 may include a set of controllable components (e.g., virtual keys shown in FIG. 4), but it should also be allowed that small regions that cannot be operated (e.g., gaps between the virtual keys in FIG. 4) are present in the controllable region.

It can be understood that the image capture apparatus 422 may be integrated on the ultrasound imaging apparatus 410, or may be present independent of the ultrasound imaging apparatus 410 and the external device 420. The image capture apparatus 422 may also perform other functions at the same time. For example, in an application scenario of remote ultrasound, the image capture apparatus 422 may also be used to acquire a facial image of the user, and the facial image is displayed in a user video window 425. Similarly, the user interface 423 further includes a remote video window 426. The user and the remote user can perform instant video communication by means of the two windows. In another embodiment, the image capture apparatus 422 may be used to acquire scanning information of the user, such as a probe manipulation method used in an ultrasound scan. It can be understood that the probe manipulation method is displayed in the user video window 425. This facilitates observation performed by the remote user, so that the remote user advises on or studies a scan.

It should be noted that the ultrasound imaging apparatus 410 in FIG. 4 is shown in the form of a portable ultrasound imaging apparatus, and may be of any other type in the art, such as a flat-bed ultrasound imaging apparatus, a bench-top ultrasound imaging apparatus, or the like. In addition, for the internal structure and program, circuits, etc., of the ultrasound imaging apparatus 410, which are not shown in FIG. 4, reference may be made to the above description of any embodiment of the present application.

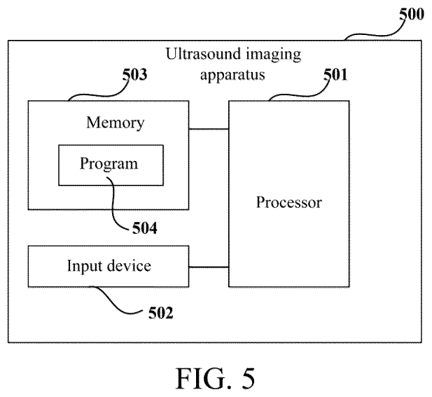

The embodiments of the present application further provide an ultrasound imaging apparatus. FIG. 5 is a schematic diagram of an ultrasound imaging apparatus according to an embodiment of the present application. As shown in FIG. 5, an ultrasound imaging apparatus 500 includes a processor 501 and an input device 502. The input device 502 may include at least one of a trackball, a keyboard, a touch pad, and a touch screen, and for implementation thereof, reference may be made to the implementation of the input device 142 in the foregoing embodiments. Details are not described herein again.

The processor 501 performs the steps of the control method according to any one of the foregoing embodiments, so as to control the input device 502, etc. For example, as shown in FIG. 5, the ultrasound imaging apparatus may further include a memory 503 coupled to the processor 501. A computer-readable program 504 is stored in the memory 503. The processor 501 may be configured to execute the program 504 to implement the control method described in the above embodiment, the contents of which are incorporated herein. Description will not be repeated herein. In addition, for implementation of the processor 501, reference may be made to the implementation of the controller circuit 102 in the foregoing embodiments, and details are not described herein again.

In addition, for the implementation of the ultrasound imaging apparatus 500, reference may also be made to the implementation of the ultrasound imaging apparatuses 110 and 410 in the foregoing embodiments, and details are not described herein again.

The embodiments of the present application further provide a computer-readable program, wherein the program, when executed, causes a computer to perform, in the ultrasonic imaging device, the control method described in the foregoing embodiments.

The embodiments of the present application further provide a non-transitory computer-readable storage medium, which is used for storing a computer program, wherein the computer program, when executed by a computer, causes the computer to execute the control method described in the foregoing embodiments.

The above embodiments merely provide illustrative descriptions of the embodiments of the present application. However, the present application is not limited thereto, and appropriate variations may be made on the basis of the above embodiments. For example, each of the above embodiments may be used independently, or one or more among the above embodiments may be combined.

The present application is described above with reference to specific embodiments. However, it should be clear to those skilled in the art that the foregoing description is merely illustrative and is not intended to limit the scope of protection of the present application. Various variations and modifications may be made by those skilled in the art according to the spirit and principle of the present application, and these variations and modifications also fall within the scope of the present application.

Preferred embodiments of the present application are described above with reference to the accompanying drawings. Many features and advantages of the implementations are clear according to the detailed description, and therefore the appended claims are intended to cover all these features and advantages that fall within the true spirit and scope of these implementations. In addition, as many modifications and changes could be easily conceived of by those skilled in the art, the embodiments of the present application are not limited to the illustrated and described precise structures and operations, but can encompass all appropriate modifications, changes, and equivalents that fall within the scope of the implementations.

1. A method of controlling an ultrasound imaging apparatus, the method comprising:
establishing a communication connection with an external device;
transmitting ultrasound data to the external device via the communication connection;
receiving, via the communication connection, user interface information about the ultrasound data, the user interface information being generated on a display interface of the external device; and
in response to generation of a switching instruction, switching an input device of the ultrasound imaging apparatus to control a user interface of the external device.

2. The method according to claim 1, wherein the generation of the switching instruction is based on at least one of the following means:
moving a cursor of the input device on a display apparatus of the ultrasound imaging apparatus out of an edge of the display apparatus towards the external device;
operating a switch button in the input device; and
determining, using an image capture apparatus, that a line of sight of a user is directed to the external device.

3. The method according to claim 2, wherein the determining, using an image capture apparatus, that a line of sight of a user is directed to the external device comprises:
acquiring the line of sight of the user using the image capture apparatus, and
based on a determination that the line of sight is directed to the external device, causing the input device to control the external device.

4. The method according to claim 3, wherein the determining, by using an image capture apparatus, that a line of sight of a user is directed to the external device further comprises:
based on a determination that the line of sight is not directed to the external device, causing the input device to control the ultrasound imaging apparatus.

5. The method according to claim 1, wherein the input device comprises at least one of a trackball, a keyboard, a touch pad, and a touch screen.

6. The method according to claim 5, wherein
the input device comprises at least one of a trackball and a touch pad, and
controlling the user interface of the external device comprises determining a controllable region of the user interface of the external device, and restricting a cursor corresponding to the trackball or the touch pad within the controllable region.

7. The method according to claim 5, wherein
the input device comprises a touch screen, and the touch screen controlling the user interface of the external device comprises:
projecting interface information of the external device onto the touch screen.

8. The method according to claim 1, wherein the display interface of the external device is configured to be touch-operable.

9. The method according to claim 8, wherein the external device comprises an image capture apparatus.

10. An ultrasound imaging apparatus, comprising:
an input device configured to receive an input;
a memory storing instructions; and
a processor configured to execute the instructions to:
establish a communication connection with an external device;
transmitting ultrasound data to the external device via the communication connection;
receive, via the communication connection, user interface information about the ultrasound data, the user interface information being generated on a display interface of the external device; and
in response to generation of a switching instruction, switch an input device of the ultrasound imaging apparatus to control a user interface of the external device.

11. The system according to claim 10, wherein the generation of the switching instruction is based on at least one of the following means:
moving a cursor of the input device on a display apparatus of the ultrasound imaging apparatus out of an edge of the display apparatus towards the external device;
operating a switch button in the input device; and
determining, using an image capture apparatus, that a line of sight of a user is directed to the external device.

12. The system according to claim 11, wherein the determining, using an image capture apparatus, that a line of sight of a user is directed to the external device comprises:
acquiring the line of sight of the user using the image capture apparatus, and
based on a determination that the line of sight is directed to the external device, causing the input device to control the external device.

13. The system according to claim 12, wherein the determining, by using an image capture apparatus, that a line of sight of a user is directed to the external device further comprises:
based on a determination that the line of sight is not directed to the external device, causing the input device to control the ultrasound imaging apparatus.

14. The system according to claim 10, wherein
the input device comprises at least one of a trackball and a touch pad, and
controlling the user interface of the external device comprises determining a controllable region of the user interface of the external device, and restricting a cursor corresponding to the trackball or the touch pad within the controllable region.

15. A non-transitory computer-readable storage medium for storing a computer program that, when executed by a computer, causes the computer to execute the method of:
establishing a communication connection with an external device;
transmitting ultrasound data to the external device via the communication connection;
receiving, via the communication connection, user interface information about the ultrasound data, the user interface information being generated on a display interface of the external device; and
in response to generation of a switching instruction, switching an input device of the ultrasound imaging apparatus to control a user interface of the external device.

16. The non-transitory computer-readable storage medium according to claim 15, wherein the generation of the switching instruction is based on at least one of the following means:
moving a cursor of the input device on a display apparatus of the ultrasound imaging apparatus out of an edge of the display apparatus towards the external device;
operating a switch button in the input device; and
determining, using an image capture apparatus, that a line of sight of a user is directed to the external device.

17. The non-transitory computer-readable storage medium according to claim 16, wherein the determining, using an image capture apparatus, that a line of sight of a user is directed to the external device comprises:
acquiring the line of sight of the user using the image capture apparatus, and
based on a determination that the line of sight is directed to the external device, causing the input device to control the external device.

18. The non-transitory computer-readable storage medium according to claim 17, wherein the determining, by using an image capture apparatus, that a line of sight of a user is directed to the external device further comprises:
based on a determination that the line of sight is not directed to the external device, causing the input device to control the ultrasound imaging apparatus.

19. The non-transitory computer-readable storage medium according to claim 15, wherein
the input device comprises at least one of a trackball and a touch pad, and
controlling the user interface of the external device comprises determining a controllable region of the user interface of the external device, and restricting a cursor corresponding to the trackball or the touch pad within the controllable region.
